(19) United States
(12) Patent Application Publication
(10) Pub. No.: US 2018/0344198 A1
(43) Pub. Date: Dec. 6, 2018

(54) METHOD AND IMAGING SYSTEM FOR OPTIMIZING A MAGNETIC RESONANCE EXAMINATION TO BE PERFORMED ON A PATIENT

(71) Applicant: Siemens Healthcare GmbH, Erlangen (DE)

(72) Inventors: Matthias Gebhardt, Erlangen (DE); Stephan Nufer, Erlangen (DE)

(73) Assignee: Siemens Healthcare GmbH, Erlangen (DE)

(21) Appl. No.: 15/992,518

(22) Filed: May 30, 2018

(30) Foreign Application Priority Data
May 30, 2017 (DE) 102017209008.1

(51) Int. Cl. A61B 5/055; G01R 33/36

(52) U.S. Cl. CPC A61B 5/055; G01R 33/543; G01R 33/36

(57) ABSTRACT

In a method for optimizing a magnetic resonance (MR) examination to be performed with an MR scanner on a patient, a token is detected that identifies a medical aid that is to be present in the MR scanner during the examination, in particular to identify an implant implanted in the patient, a medical device to be used during the MR examination or a medical prosthesis worn by the patient. At least one restriction for the planned MR examination is determined in a computer as a function of the token.

### BACKGROUND OF THE INVENTION

### Field of the Invention

The present invention concerns a method for optimizing a magnetic resonance (MR) examination to be performed on a patient, as well as an MR examination system and a non-transitory, computer-readable data storage medium that implement such a method.

### Description of the Prior Art

For MR examinations, there is currently no binding standard for the detection and presetting of patient implants. An operator of an MR apparatus therefore cannot obtain any reliable confirmation as to whether the implant is suitable for the scheduled MR examination or not.

On the Internet there are a few websites or other sources that list various implants and provide details with regard to their MR examination compatibility. However, these databases are neither complete nor certified by an institution, and in addition, these databases provide no guarantee of accuracy. In order to obtain access to these databases, it is necessary to hold up an Internet-enabled device in the vicinity of the MR apparatus, which for reasons of data protection is usually not on site. A further reason for forgoing such websites is that the examination protocols and options that are proposed on the website must be understood and correctly selected by the operator of the MR apparatus. A further problem is that the operators of the MR apparatus do not usually have a deep understanding of the underlying physics and do not feel confident to examine a patient with an implant, for both technical and psychological reasons.

DE 10 2014 209 649 discloses a method wherein a control console is locally separated from the MRI scanner and consequently patient data can be recorded prior to an MR examination. The MRI scanner and the control console are designed so that patient data also can be displayed.

### SUMMARY OF THE INVENTION

An object of the present invention is to provide a method for MR examinations that eliminates the aforementioned disadvantages and creates the possibility of examining a patient with an implant or another medical aid safely, quickly and easily.

According to the invention, the method for optimizing a magnetic resonance examination to be performed on a patient having an implant, using an MR apparatus (scanner) has the following steps.

A token that identifies a medical aid that is to be present in the scanner during an MR scan is brought into the field of view of a taken identifier, such as a reader or detector or the MR apparatus operator, in order to identify an implant implanted in the patient, a medical device to be used during the MR examination, or a medical prosthesis worn by the patient. Automatic determination of at least one restriction for the planned MR examination is then determined in a computer in communication with the token identifier, as a function of the identified token.

In effect, the method changes the state of the MR scanner that is to be used to implement the MR examination of the patient, by placing the MR scanner in a state that is dependent on or embodies said at least one restriction, in order to then conduct the MR examination according to that at least one restriction.

The token for the identification of a medical aid to be used on or in the MR scanner typically has a machine-readable code, which can be a barcode, a QR code or even a type number or serial number, preferably in ASCII code, in particular letters and numbers. Preferably, identification can take place by an optical detection, which can be applied on a portable computer. The detection of the token takes place by an operator of the MR apparatus, or automatically. Detection preferably takes place with a portable computer but can also be performed manually by the operator of the MR apparatus (scanner). For example, the operator enters the token manually in the database and/or the portable computer. In a preferred embodiment, the method can also be integrated and/or executed in an ERP system or a patient management system.

A medical aid can be an implant implanted in a patient, a medical device to be used during the MR examination or a medical prosthesis worn by the patient. Other objects that are envisaged during the MR examination in or on the MR scanner are also conceivable. A multiplicity of medical aids is conceivable, including a hip implant, an artificial leg, a cardiac pacemaker, an arm splint, a ventilator, and in general, all aids having relevance to the operation of the MR scanner with regard to magnetic qualities.

The token for identification and/or the restrictions are individually determined for each medical aid and are preferably certified by an institution such as the manufacturer of the medical aid. The automatic determination of at least one restriction results in the determination of a restriction for the planned MR examination as a function of the token that identifies a medical aid to be used on or in the MR scanner. The restriction relates to the medical aid to be used on or in the MR scanner and to the planned MR examination. The restriction preferably describes a property of the MR examination which must be adjusted so as to enable the successful performance of the MR examination. The restriction describes a physical variable or a setting parameter that is typical for the MR scanner.

An advantage of this method is that the detection of a medical aid takes place quickly and free of errors, with computer support, even if untrained personnel question the patient about his or her implant and/or read out the certificate or the token. A further advantage is that medical aids are internationally prepared according to the same standards and/or according to the proposed invention for an MR examination.

For example, the method is performed when a patient with an implanted implant visits a medical institution where the patient is requesting to undergo an MR examination. A member of staff of the medical institution reads out the token for identification of the implant, of which the patient is in possession, with the use of a portable computer, and thus the computer automatically determines the examination restrictions for the planned MR examination of the specific patient.

Preferably, the detection of the token for identification of the medical aid takes place by means of a machine-readable code printed on a certificate, which is read into the computer via a camera of the portable computer. The certificate can be printed on a piece of paper or can be available in the form of a plastic card. A certificate is preferably an attestation issued by the manufacturer of the medical aid, or a certification institution of medical aids that the medical aid worn by the patient, or the medical aid that is to be present in the scanner during the MR examination, is the medical aid that is described on the certificate. A machine-readable code printed on the certificate is preferably a barcode or QR code or a similar arrangement, which can be entered easily as an input, e.g. by a portable computer with a camera. In a preferred embodiment, a machine-readable code can be identified not only by optical detection but also by an electromagnetic, infrared-based or other typical transfer methodology in the field of IT technology. The advantage of this embodiment is that in the identification of a token of a medical aid by the automated method and computer support, no input errors can take place, and there is a significant reduction in errors, as no human errors can occur. Alternatively, the certificate can be created in digital form, e.g. as a two-way security token that is transmitted to the portable computer with the use of digital transmission techniques such as e-mail or the like. For example, a patient brings the certificate with him or her to the scheduling of the MR examination to the actual MR examination, the certificate having a machine-readable code, for example, a QR code, so the certificate can easily be scanned in by the operator of the MR apparatus with the use of a camera of a portable computer, for example, a tablet computer.

In an embodiment, the restriction is determined by making a query to a local and/or network-based database. In other words, the restrictions in a local or network-based database triggered by such a query are determined. This query is triggered by a (preferably) portable computer and/or the console of an MR apparatus and/or by the MR scanner itself, which seeks to determine restrictions for the planned MR examination. The restrictions of the planned MR examination are determined as a function of the token that is transmitted to the local or network-based database as part of the query. This has the advantage that all the restrictions can be compiled and maintained in a local and/or network-based database and administrative expenses are thus saved. The local database can be a database that e.g. is provided in the MR apparatus. Likewise, the local database can be situated outside the MR scanner, but in its immediate vicinity. In a further embodiment, a medical institution has a database with which all the MR scanners of the medical institution are connected, so consequently updating of the database need only take place at one centralized point. A network-based database is a database that is connected to one or more MR apparatus of a local network or the Internet. A network-based database may likewise be a cloud solution, wherein the database is spread over several different server systems.

In the embodiment of the method, wherein a query is made, the token is received by the local and/or network-based database, restrictions are selected as a function of the token, and the restrictions are sent to the MR scanner.

Preferably the tokens are received fully automatically by the local and/or network-based database, with the connection between the local or network-based database and the portable computer being a direct connection or a connection including the intermediate step via a network. As a function of the token, the database selects restrictions for the planned MR examination that are suitable for an examination with the medical aid, which is in or on the MR scanner during the MR examination. Subsequently, the local and/or network-based database sends the restrictions, which were determined and selected, to the MR device. These steps have the advantage that the restrictions for the planned MR examination can be quickly and easily determined and there is a low probability of error. In a specific example, the local and/or network-based database receives a token of a hip implant for which the restrictions are selected as a function of the token and then these restrictions are sent to the MR scanner.

When the aforementioned query is unsuccessful, the most conservative selection of restrictions is selected for the MR examination. If no restrictions are specified in the local and/or network-based database for the token inside the query process, this query is to be interpreted as unsuccessful. In this case, the local and/or network-based database sends information to the MR scanner that no restrictions could be ascertained for the planned MR examination. The most conservative selection of restrictions describes the settings of the MR scanner, on which the planned MR examination is to be performed, which ensure an imaging process in which the RF and gradient performance of the MR scanner are reduced as much as possible, in particular by only considering the type of medical aid, e.g. as a hip implant. Expediently, a user can undertake a manual input of the type of medical aid (without precise identification) in order to thus enable an MR examination, even if this has the most conservative examination attributes. The advantage of this embodiment is that the likelihood of problems during the MR examination is significantly reduced, because the settings of the MR scanner are already geared to an examination with a medical aid.

In a further embodiment of the method, the restriction of the medical aid means that the MR examination is not possible. The conclusion that an MR examination is not possible means in this context that, with the use of the ascertained token for the medical aid, which is to be used in or on the MR scanner during the MR examination, a recommendation was ascertained from the local and/or network-based database that prevents the proposed MR examination. An advantage of this embodiment is that an MR examination with a medical aid is not started and there is therefore a significantly lower risk of injuring a patient and/or problems during the MR examination. A further advantage is that through forward-looking planning, utilization of the MR scanner increases significantly as patients can be examined before the actual MR examination with regard to their suitability for the MR examination depending on the medical aid.

With the use of the patient's smartphone and/or a portable computer, the patient can detect the token on the certificate for identification of the implant implanted in the patient, in particular, in temporal connection with appointment allocation, or at least 5 to 24 hours prior to the planned examination. In this way, the token can be detected long before the actual MR examination and the allocation of appointments by the medical institution optimized.

For example, for the planned MR examination a ventilator which is positioned on the MR scanner was envisaged but, by querying the token of the ventilator, it became apparent that this ventilator is not suitable for the planned MR examination.

In a preferred embodiment, if the querying of the restriction is unsuccessful, an order for detection of the restriction as a function of the token and the medical aid is triggered. This order is a notification that is triggered by the local and/or network-based database which is directed at the manufacturer of the medical aid, and asks the latter to update restrictions for an MR examination as a function of the token and/or the medical aid in the local and/or network-based database. The detection of restrictions is understood to mean that minimum and maximum values and preferred areas of the physical variables are fed into the local and/or network-based database for the specific physical variables of an MR examination. This has the advantage that the local and/or network-based database automatically takes care of obtaining the restrictions of the medical aid for MR examinations and thus reduces errors, as well as significantly reducing effort for qualified personnel. In an example, with the use of the token of the medical aid, for example, a hip implant, no restrictions and/or false restrictions were ascertained in the local and/or network-based database, triggering an order for the detection of the restrictions of the hip implant by the manufacturer of the hip implant.

In another embodiment, the token for identification of the medical aid and the restriction during the query are encrypted and, in particular, given a checksum. Encrypting the query in this embodiment means encrypting both the token for identification of the medical aid as well as the restriction of the planned MR examination with a typical crypto-encryption method, for example, PGP or SSL encryption, and preferably providing these encryptions with a checksum that enables both the completeness of the encryption to be examined as well as the restriction of the planned MR examination in terms of its plausibility. This has the advantage that patient-relevant data can be efficiently protected and in addition, a further source of errors can be eliminated because the checksum automatically monitors all the transmitted data. A further advantage of providing encryption with a checksum is therefore ensuring the accuracy and reliability of the data, in particular if the data have a digital signature. In an exemplary embodiment, the token for identification is encrypted with an SSL encryption method and provided with a checksum and transmitted to the local and/or network-based database, decrypted there, and the checksum examined and then the restriction for the planned MR examination ascertained. The restrictions are encrypted again using an SSL method and given a checksum and transferred to the MR scanner where encryption and examination of the transmitted restrictions takes place.

In another embodiment, the detection of the token for identification in an active implant takes place by wireless transmission between the active implant and a portable computer. An active implant means an implant that can communicate with a portable computer via a communication unit by wired or wireless transmission. In wired and/or wireless transmission between the active implant and the portable computer, the transmission method preferably is a type of transmission typical in the medical technology sector, such as ultrasound transmission, electromagnetic fields or Near Field Communication. This has the advantage that false identification cannot take place because the implant that is implanted in the patient does not supply any erroneous information and in addition, no erroneous inputs can be made by, for example, medical experts. In an exemplary embodiment, a patient with an active implant visits a medical institution for MR examinations and, during the preparation of this MR examination, the active implant is read with the use of ultrasound technology so as to obtain restrictions for the planned MR examination.

Preferably, the active implant is designed such that, in response to each semi-public request, it sends the same data packet containing, for example, the manufacturer, the precise type of implant and/or the serial number. A semi-public request means a data-technological stimulation of the active implant, whereupon the active implant sends a data packet containing information in response, for example, the manufacturer of the implant, the precise type of implant and/or the serial number of the implant. The request can be started, for example, by a portable computer or another readout device. Preferably, the active implant always gives the same data packet in response to a semi-public request. This embodiment has the advantage that the specific characteristics of the active implant can be simply read out and no patient-relevant information is transmitted and that no manipulation of the active implant can take place. In an example, with the use of a tablet computer a request is made to the active implant and the data packet received by the tablet computer and transferred to the MR scanner.

In a preferred embodiment, the active implant transfers the restriction to the portable computer upon request, wherein the restriction is compared with the local and/or network-based database. In this embodiment, the implant stores all the restrictions for an MR examination in a memory that is provided in the active implant and can transmit this data to a portable computer via a communication unit. The portable computer compares the restrictions ascertained in this way with the use of a local and/or network-based database to thus enable an examination of the transmitted data to be undertaken. This has the advantage that the complete set of restrictions for an MR examination can be read out in one method step and simultaneously there is a reduction in errors because of comparison with the local and/or network-based database. For example, a medical expert initiates a request by means of Bluetooth with the use of a portable computer to the active implant, which transmits the restrictions for a planned MR examination to the portable computer and this compares the restrictions with the use of a network-based database and corrects these, if necessary.

The method can include the further step of calculation of at least one examination attribute as a function of the restriction for the planned MR examination.

The calculation of at least one examination attribute means that the restriction is an area of physical variables for the planned MR examination and the calculation of an examination attribute calculates a concrete value for the planned MR examination. For example, a maximum duration of the examination is derived from the maximum specific absorption rate or, for example, a specific MR scanner selected as a function of the transmitter and/or receiver coils available in the medical institution.

Preferably, the method includes the further step of determination of an MR sequence as a function of the restriction and/or the examination attribute and presetting of this MR sequence on the MR device.

The determination of an MR sequence involves the selection and adjustment of an MR sequence as a function of the restrictions which constitute the physical variables for the MR examination and/or of the examination attribute which, for example, specifies and calculates a concrete value for a physical variable for the planned MR examination and presets these calculated values on the MR scanner for the planned MR examination, in particular the MR sequence. This has the advantage of bringing about improved image quality of the MR examination without qualified personnel being required for input, which in turn leads to a cost/time savings.

Preferably, the method includes the further step of storage of the token for identification of the medical aid, the restriction and the examination attribute.

Storage in this embodiment means the storage of all characteristic values and determined values of the token for identification of the medical aid, the restrictions and the examination attribute on a data carrier which is provided in the MR scanner, or in a local or network-based database. This has the advantage that, for a renewed determination of the MR sequence or another change in the MR scanner, all the values need not be determined and/or entered again but are already retained to save processing time.

Preferably, all of the aforementioned steps are automatically executed. Automatically executed means that the method either executes itself independently with computer support or a user initiates the first method step and the method is automatically run as far as the last method step and then the result of the last method step is presented to the user.

Preferably, the restriction is one or more of the following parameters of the MR examination:
Maximum specific absorption rate (SAR)
Maximum power of the magnetic RF field
Average of the magnetic RF field, in particular B1+RMS,
Maximum value of the magnetic RF field, in particular B1+Peak
Maximum value of the ratio between the change of amplitude of the magnetic field and the temporal change of the magnetic field (dB/dt Peak)
Average of the ratio between the change of amplitude of the magnetic field and the temporal change of the magnetic field (dB/dt rms)
Maximum slew rate (e.g. in T/m/s)
Maximum value of the spatial gradients (e.g. in T/m)
Maximum/minimum dimension of a “field of interest”
Restrictions of the permitted measurement sequence (e.g. no EPI)
Restrictions on contrast agent indications
Types of transmitter/receiver coils.

Preferably, the aforementioned parameters are specified in a physical variable typical for this purpose, in particular, a unit that is internationally recognized for these parameters.

The invention also uses a database configured to provide at least one restriction for an MR examination as a function of the token for a query, the database including at least one token of a medical aid, wherein the database can be designed as a local or network-based database. The database is preferably an embodiment customary in IT such as, for example, an SQL database or a cloud-based database which is executed on one or more servers. The advantage hereby is that the database is preferably available for many users of the MR scanners and a uniform examination can thus be ensured.

The present invention also encompasses a non-transitory, computer-readable data storage medium encoded with programming instructions that, when the storage medium is loaded into a portable computer, cause the portable computer to implement any or all of the embodiments of the method according to the invention, as described above.

The invention also encompasses a portable computer configured to detect a token for the identification of a medical aid, and configured to ascertain at least one restriction for an MR examination, wherein the portable computer has a camera configured to evaluate machine-readable code, and/or wherein the portable computer has a wireless interface configured to make a request to an active implant and to receive and interpret a response from the active implant. The portable computer is preferably a tablet computer, a smartphone, a handheld device or a laptop which is directly connected to the MR scanner. The portable computer can also be part of the MR scanner. The portable computer has a camera and/or an optical sensor which is designed to evaluate and/or detect machine-readable code and process this. In an alternative embodiment, the portable computer can also be designed as a stationary computer, in particular, a desktop PC. The wireless interface of the portable computer is preferably a WLAN, Bluetooth, infrared or other wireless transmission technology which the portable computer can execute. In some embodiments, the portable computer can make a request to an active implant and receiving and interpreting a response to the request to the active implant. The advantage of this embodiment is that information with regard to the MR examination can be quickly read into the MR scanner by the portable computer and a significant reduction in errors thus takes place, and an increase in the efficiency of the overall MR examination process is obtained.

All the features described with regard to the method according to the invention can be executed and applied by the database, the data carrier and the portable computer.

### BRIEF DESCRIPTION OF THE DRAWINGS

FIG. 1 schematically illustrates the method for optimizing an MR examination according to an exemplary embodiment of the invention;

FIG. 2 schematically illustrates the method for optimizing an MR examination according to another exemplary embodiment of the invention.

FIG. 3 schematically illustrates the method for optimizing an MR examination according to another exemplary embodiment of the invention.

### DESCRIPTION OF THE PREFERRED EMBODIMENTS

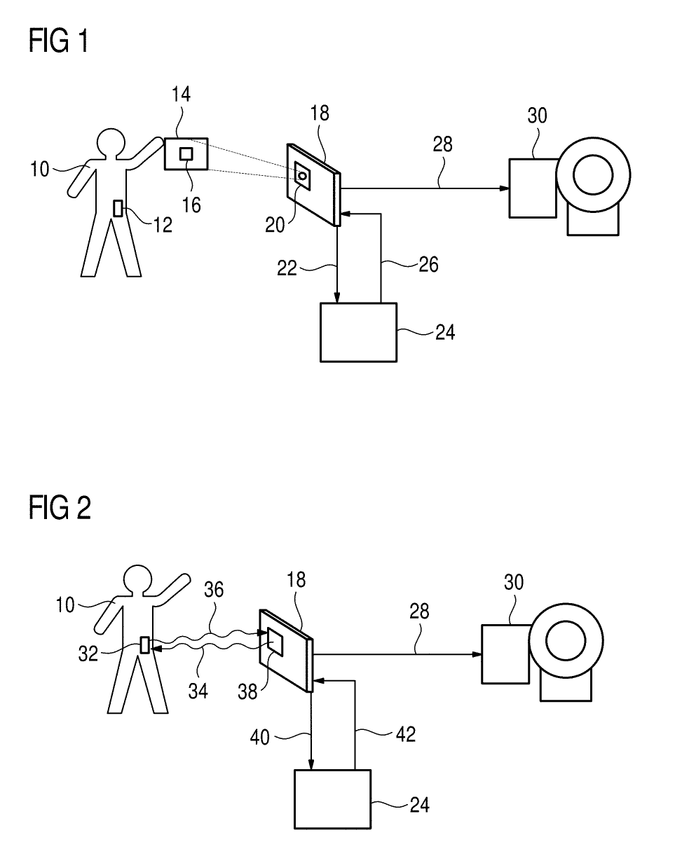

FIG. 1 shows a patient (10) who has an implant (12) implanted in his or her body. The patient (10) has a certificate (14), for example in the form of a card showing information about the patient's implant (12). The certificate (14) has a machine-readable code and/or token which contains information for identification of the implant (12). The portable computer (18) has a camera (20) configured to detect the token (16). As a function of the token (16), the portable computer initiates a query (22) to the database (24) which can be a local or network-based database. The database (24) sends restrictions (26) regarding a planned MR examination to the portable computer (18). The portable computer (18) transmits the restrictions (28) to the MR scanner (30). In a preferred embodiment, the database (24) transmits the restrictions (26) directly to the MR scanner (30)

FIG. 2 shows a patient (10) with an active implant (32) which was implanted in the patient. In this embodiment, the portable computer (18) has a wireless communication unit (38) which makes a wireless request (34) to the active implant (32). As a function of this request (34) the active implant sends a response (36) to the wireless communication unit (38) of the portable computer (18). The response (36) includes restrictions for MR examinations for the active implant (32) and preferably additional information such as the manufacturer and the serial number. The portable computer (18) compares the restrictions that were specified in the response (36) with the database (24) in a comparison step (40). If the values in the database (24) match the values of the response (36), the database reports positive feedback (42) to the portable computer (18). The portable computer (18) transmits the restrictions (28) to the MR scanner (30). In a preferred embodiment, the database (24) transmits the restrictions (26) directly to the MR scanner (30).

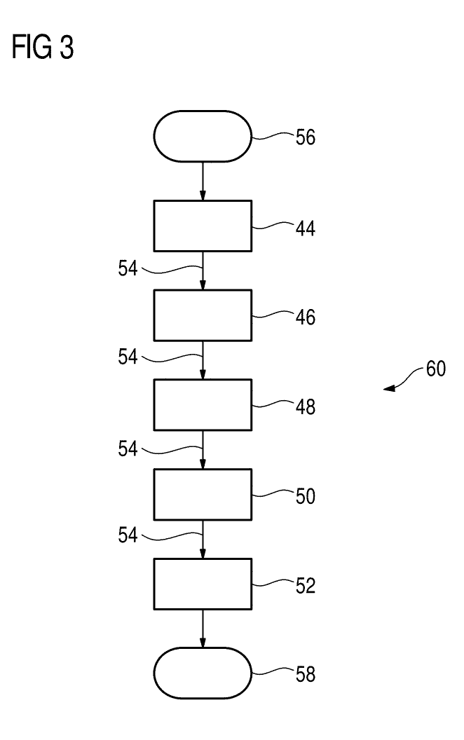

FIG. 3 schematically shows the flow of the method steps a.) to e.), which can be performed in the sequence shown or in any sequence desired. The method (60) is initiated by the trigger (56). The trigger (56) can be automatically self-actuating, or activated by a manual input. In the embodiment described, the method starts with the step a.) (44). After the step a.) (44), the step b.) (46) is performed, wherein a transmission (54) with an optional encryption of the information takes place between the two steps. Then following the step b.) (46), the steps c.) (48), d.) (50) and e.) (52) are performed. Between each step transmission (54) takes place, which may be encrypted. The method (60) provides the result (58) after successful performance.

Although modifications and changes may be suggested by those skilled in the art, it is the intention of the Applicant to embody within the patent warranted hereon all changes and modifications as reasonably and properly come within the scope of the Applicant's contribution to the art.

1. A method for placing a magnetic resonance (MR) scanner of an MR apparatus in a state to perform an examination on a patient, said method comprising:
prior to implementing an MR examination of a patient by operation of the MR scanner, detecting a token that identifies a medical aid that will be present in the MR scanner during the examination;
providing a designation of the medical aid identified by the token into a processor and, in said processor, automatically determining at least one restriction for said examination dependent on the medical aid identified with the token; and
providing an electronic representation of said at least one restriction to said MR scanner and changing a state of said MR scanner, so as to embody said at least one restriction, in order to place said MR scanner in a state for conducting said examination of the patient.

2. A method as claimed in claim 1 wherein said identification of said medical aid is an identification of an implant implanted in the patient, a medical device to be used during the examination, and a medical prosthesis worn by the patient.

3. A method as claimed in claim 1 comprising detecting said identification of the medical aid by reading a machine-readable code printed on a certificate, using a camera of a portable computer.

4. A method as claimed in claim 1 comprising determining said restriction by making a query from said portable computer to a local database or a network-based database in which electronic designations of different tokens are stored.

5. A method as claimed in claim 4 comprising, in said query:
receiving said token from said portable computer at said local database or said network-based database;
selecting said at least one restriction dependent on the received token; and
sending said restriction from said local base or said network-based database to said MR scanner.

6. A method as claimed in claim 4 comprising, if said query is unsuccessful, automatically defaulting to select a most conservative restriction for said examination.

7. A method as claimed in claim 6 wherein said most conservative restriction is a preclusion from implementing said examination.

8. A method as claimed in claim 4 comprising, if said query is unsuccessful, providing an order to said portable computer to detect a restriction dependent on said token.

9. A method as claimed in claim 3 comprising encrypting said token and said restriction during said query.

10. A method as claimed in claim 1 comprising detecting said token by wireless transmission between said implant and a portable computer.

11. A method as claimed in claim 10 comprising, from said implant, sending a same data packet in response to a wireless inquiry from said portable computer, said data packet comprising at least a designation of a manufacturer of said implant, a type of said implant, and a serial number of said implant.

12. A method as claimed in claim 11 comprising transmitting said restriction from said implant to said portable computer upon a request from said portable computer, and comparing said restriction via said portable computer with restriction stored in a local database or a network-based database.

13. A method as claimed in claim 1 comprising, in said computer, calculating at least one attribute of said examination as a function of said restriction, and including said attribute in said state of said MR scanner for implementing said examination.

14. A method as claimed in claim 1 comprising, in said computer, determining an MR sequence as a function of said restriction and setting said MR scanner to execute said determined MR sequence in said state of said MR scanner for executing said examination.

15. A method as claimed in claim 1 comprising storing said token together with said restriction in a memory accessible via said computer.

16. A method as claimed in claim 1 wherein said restriction is at least one of a maximum specific absorption rate, a maximum power of an RF field, an average of an RF field, a maximum value of an RF field, a maximum value of a ratio between a change in amplitude of a magnetic field and a temporal change of said magnetic field, an average of a ratio between said change, a maximum or minimum slew rate, a maximum or minimum value of spatially encoding gradients, a maximum or minimum dimension of a region of interest of the examination subject, a maximum or minimum value for a parameter of an MR data acquisition sequence, a restriction as to contrast agent administration, a type of RF transmitter coil to be used, and a type of RF receiver coil to be used.

17. A non-transitory, computer-readable data storage medium encoded with programming instructions, said storage medium being loaded into a computer associated with a magnetic resonance (MR) scanner of an MR apparatus, said programming instructions causing said computer to:
prior to implementing an MR examination of a patient by operation of the MR scanner, detect a token that identifies a medical aid that will be present in the MR scanner during the examination;
receive a designation of the medical aid identified by the token and automatically determine at least one restriction for said examination dependent on the medical aid identified with the token; and
provide an electronic representation of said at least one restriction to said MR scanner that changes a state of said MR scanner, so as to embody said at least one restriction, in order to place said MR scanner in a state for conducting said examination of the patient.

18. A portable computer for placing a magnetic resonance (MR) scanner of an MR apparatus in a state to perform an examination on a patient, said computer comprising:
a detector that, prior to implementing an MR examination of a patient by operation of the MR scanner, detects a token that identifies a medical aid that will be present in the MR scanner during the examination;
a processor that, dependent on a designation of the medical aid identified by the token is configured to automatically determine at least one restriction for said examination dependent on the medical aid identified with the token; and
said processor being configured to provide an electronic representation of said at least one restriction to said MR scanner that changes a state of said MR scanner, so as to embody said at least one restriction, in order to place said MR scanner in a state for conducting said examination of the patient.
